(19) United States
(12) Patent Application Publication
(10) Pub. No.: US 2016/0287333 A1
(43) Pub. Date: Oct. 6, 2016

(54) HAND HELD TREATMENT DEVICE

(71) Applicants: Euan MORRISON, Harston Cambridgeshire (GB); SAGENTIA LIMITED, Cambridgeshire (GB)

(72) Inventor: Euan MORRISON, Harston Cambridgeshire (GB)

(73) Assignee: Sagentia Limited, Harston Cambridgeshire (GB)

(21) Appl. No.: 15/037,655

(22) PCT Filed: Nov. 18, 2013
(86) PCT No.: PCT/GB2013/053041

§ 371 Date: May 18, 2016

(30) Foreign Application Priority Data
Nov. 19, 2012 (GB) 1220795.7

(51) Int. Cl. A61B 18/20

(52) U.S. Cl. CPC A61B 18/203; A61B 2018/00476

(57) ABSTRACT

A handheld device has an array of light sources, for example light emitting diodes. The array is divided into different groups and selection circuitry selects a group of energy sources to be driven at any given time. Control circuitry controls the selection so that the different groups of light sources are driven at different times.

The invention relates to a device and method for delivering light energy to the skin of a user for treatment purposes.

Selective photothermolysis is a well-known technique for damaging or destroying hair follicles in the skin using visible and near infra-red light.

Originally the technique was developed for the professional dermatology market. Recently, a number of home use devices have appeared on the market. Almost all of these devices use intense pulsed light (IPL) to deliver a short pulse of light which penetrates the skin and causing heating of the hair follicles to a level where the hair follicle is damaged or destroyed.

The technique of selective photothermolysis relies on the use of short pulse lengths and high energy densities to deliver sufficient energy to the hair follicle to cause damage. The technique only works with hair follicles which absorb higher levels of light energy than the surrounding skin tissue. The technique works well with dark hair and light skin. It is less effective for light coloured hairs and for dark skin types. The pulse duration is an important factor. Typically IPL based hair removal devices use pulse lengths between about 1 ms to about 40 ms. The pulse length is selected so that it is less than the thermal relaxation time constant of the hair follicle in the skin. When a pulse of light is applied to the skin which has a pulse length of less than the thermal relaxation time of the hair follicle in the skin, and the hair follicle absorbs more light per unit volume than the surrounding skin tissue, the hair follicle will temporarily increase in temperature compared to the surrounding skin tissue. The process of selective thermolysis for hair removal relies on elevating the hair follicle to a temperature at which sufficient damage occurs within the hair follicle cells to prevent growth (estimated at 60-70° C.).

The technique of hair removal using selective thermolysis was first developed for the professional dermatologists and beauty salons using intense pulsed light (IPL) delivered by xenon flash tubes. More recently a number of home use devices have appeared on the market which use the same basic technology but are typically equipped with smaller flash lamps. The size of the flash lamp drives most of the cost of the equipment—in order to make the product sufficiently low cost for the home use market, the flash lamps have been reduced in size to cover areas of typically 2 cm² and up to no more than 6 cm². Manufacturers of such products include Philips, Remington, Home Skinovations and Cyden.

All of the currently available products have very similar specifications—they all use intense pulsed light (IPL), delivered by Xenon flash tubes, the energy density delivered is typically in the region of 5-8 J/cm2 and the pulse length is around 1-30 ms. The light which is generated by the IPL lamp is typically filtered to remove the short wavelength components (<450-500 nm). This light does not penetrate the skin and would otherwise result in burning of the upper skin layers. In addition, removing the UV/blue component of the light is important from an eye safety point of view.

IPL devices which use Xenon flash lamps suffer from a number of well-known problems. The flash lamp needs to be designed to withstand a significant amount of thermal shock and as a result the lifetime of a standard flash lamp is typically less than 1000 flashes. Longer lifetimes can be achieved (>10,000 flashes) but these flash lamps are significantly more expensive to manufacture.

The pulse duration of a flash lamp is difficult to control. Much of the cost in an IPL flash lamp is in the drive electronics. The drive electronics typically consist of a large capacitor which is charged from a mains derived DC voltage. This capacitor is discharged periodically through the flash lamp to create the light pulse. An uncontrolled discharge through the flash lamps which are used in home use IPL products will generate a pulse length of about <1 ms.

The flash lamp generates a source of light which is linear along the tube—the light source dimensions may be typically 20 mm in length and about 1 mm in diameter. In order to generate a uniform illumination profile across the skin surface to be treated by the IPL device, an optical system (reflector or diffuser) is used. This optical system, in addition to the light filtering (mentioned earlier), introduces loss into the system and as a result the flash lamp and surrounding components heat up during use. This limits the pulse repetition rate of the lamp to typically 1 pulse every 1-3 seconds. Thermal management of the flash lamp unit to deal with the losses also adds cost and complexity to the device.

The present invention aims to provide an alternative light treatment device. Preferred embodiments provide a semiconductor light source based treatment device that can be used, among other things, to damage or destroy hair follicles in the skin.

Aspects of the present invention are set out in the independent claims and preferred features are set out in the dependent claims.

According to one aspect, the present invention provides a device for providing energy to the skin of a user, the device comprising: an array of semiconductor light sources; a power supply circuit for providing electrical energy to the semiconductor light sources; and a controller for controlling the operation of the semiconductor light sources, to cause the semiconductor light sources to emit light in pulses; and wherein: the array of semiconductor light sources and the power supply circuit are configured such that the array of semiconductor light sources emit light at an energy density greater than 0.5 J/cm².

Typically, the array of semiconductor light sources and the power supply circuit are arranged such that the energy density of the light emitted by the array is substantially uniform over the user's skin. The array of semiconductor light sources may emit light over an area between 1 cm² and 100 cm². One or more and preferably all of the semiconductor light sources are configured to emit single wavelength light or narrow-band light having a bandwidth between 1 nm and 100 nm. The semiconductor light sources comprise one or more of: light emitting diodes (LEDs), laser diodes, VCSELs and superluminescent diodes.

In some embodiments, the semiconductor light sources emit light having a skin penetration depth of more than 1 mm, and preferably more than 2 mm. In such embodiments, the semiconductor light sources may emit light having a wavelength that is longer than 600 nm, and preferably longer than 650 nm. Typically, the semiconductor light sources emit light having a wavelength that is shorter than 900 nm, and preferably shorter than 750 nm. In one preferred embodiment, the semiconductor light sources emit light having a wavelength of approximately 650 nm. This is preferred due to the availability of efficient light sources operating at this wavelength.

The array of semiconductor light sources and the power supply circuit may be configured, in some embodiments so that the array of semiconductor light sources emit light at an energy density greater than 1.5 J/cm² and less than 5 J/cm² and preferably at an energy density of approximately 1.8 J/cm².

In some embodiments, the semiconductor light sources have a packing density that is greater than 1 source per 15 mm² and less than 1 source per 2 mm².

The array of semiconductor light sources can be arranged in a plurality of groups and the controller can control the driving of the semiconductor light sources in each group so that the semiconductor light sources in the different groups are driven during different (completely separate or partially overlapping) time intervals. In a preferred embodiment, the controller is arranged to control the driving of the semiconductor light sources so that no two adjacent groups emit light sequentially, as this helps with heat dissipation. The controller can control the driving of the semiconductor light sources so that semiconductor light sources are driven periodically, for example, approximately once every 200 ms. The controller may control the driving of the semiconductor light sources so that the semiconductor light sources are driven to emit pulses of radiation having a pulse duration of approximately 30 to 50 ms.

A sensor may be provided to monitor at least one of the temperature of the skin surface and the shade of the skin surface.

Typically, the device is handheld, although it may have an associated docking station if desired.

One of the advantages that the invention provides is that no focussing optical element is required in front of the array of light sources. The sources are able to provide the desired energy density at the skin surface (which can be sufficient for hair removal by selective photothermolysis), without the use of such focussing optics, which can increase the size and cost of the device.

The present invention also provides a device for providing energy to the skin of a user, the device comprising: an array of light sources arranged in a plurality of groups; selection circuitry for selecting a group of light sources; a power supply circuit for providing electrical energy to the selected group of light sources; and a controller for controlling the selection circuitry to select different groups of the light sources at different times so that different groups of energy sources receive power from the power supply circuit during different time intervals. In this case, the light sources can be any light sources.

One or more (and preferably each) of the groups of light sources may comprise at least one string of serially connected light sources. This is particularly useful where the light sources are semiconductor light sources, such as LEDs.

The invention also provides a method of providing energy to a surface of the skin, the method comprising: providing an array of semiconductor light sources; providing electrical energy to the semiconductor light sources; controlling the operation of the semiconductor light sources, causing them to emit light in pulses at an energy density greater than 0.5 J/cm²; and directing the radiation emitted from the EM energy sources towards the surface of the skin.

The invention also provides a method of providing energy to a surface of the skin, the method comprising: providing an array of light sources arranged in a plurality of groups; selecting a group of light sources; providing electrical energy to the selected group of light sources; controlling the selecting to select different groups of the light sources at different times so that different groups of light sources receive electrical energy at different times; and directing the light emitted from the light sources towards the surface of the skin.

These and other features and aspects of the invention will become apparent to those skilled in the art from the embodiments described below.

In order that the invention may be more readily understood, a description will now be given of a number of exemplary embodiments that are explained with reference to the accompanying drawings, in which:

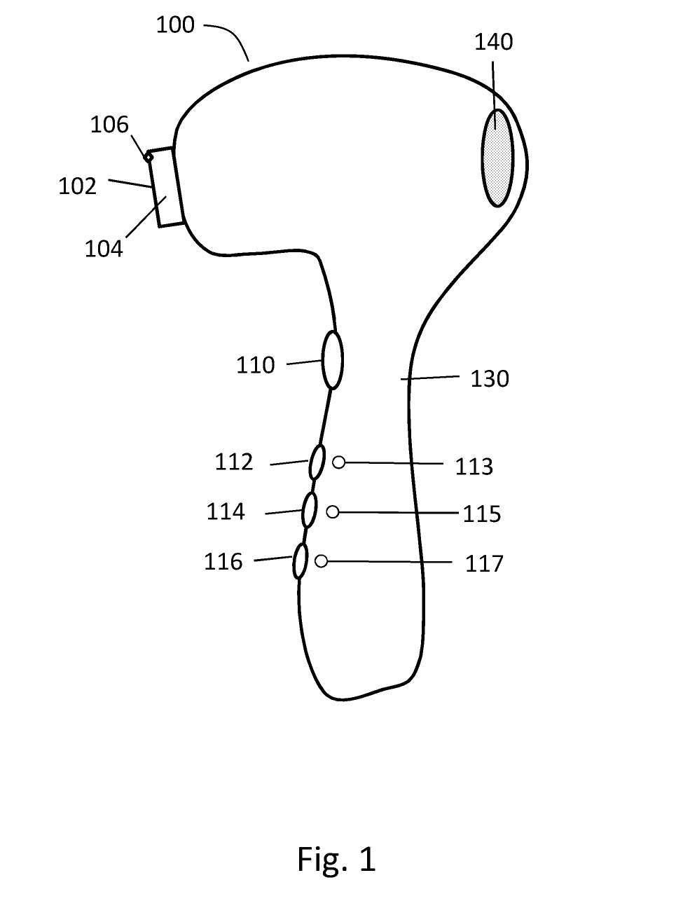

FIG. 1 shows a side view of a handheld device for providing energy to a surface of the user's skin;

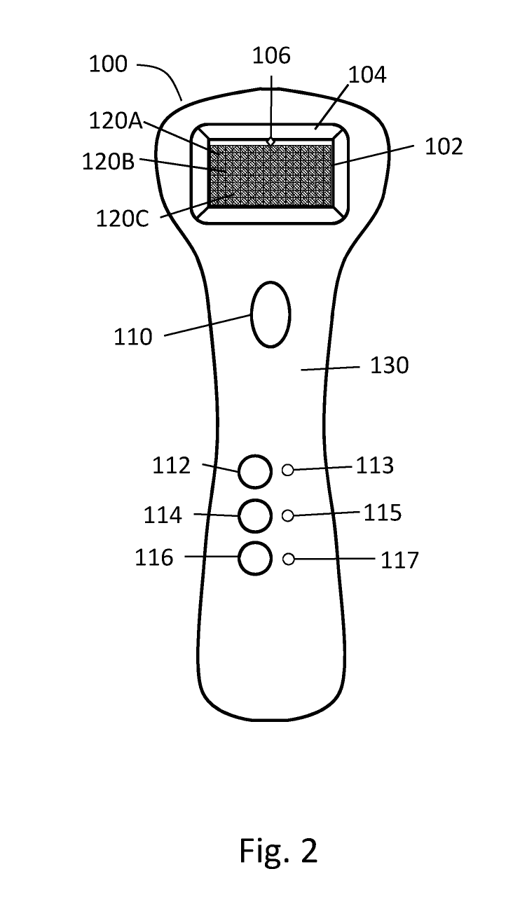

FIG. 2 shows a front view of the device of FIG. 1;

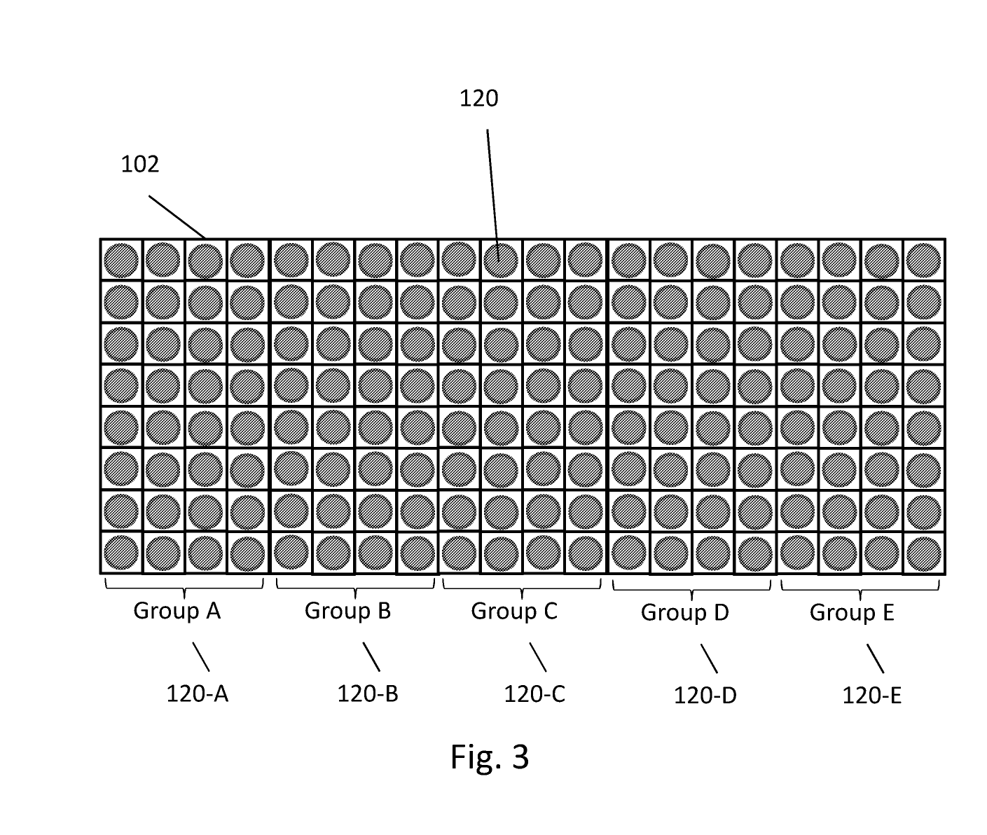

FIG. 3 shows an array of LEDs used in the device shown in FIGS. 1 and 2;

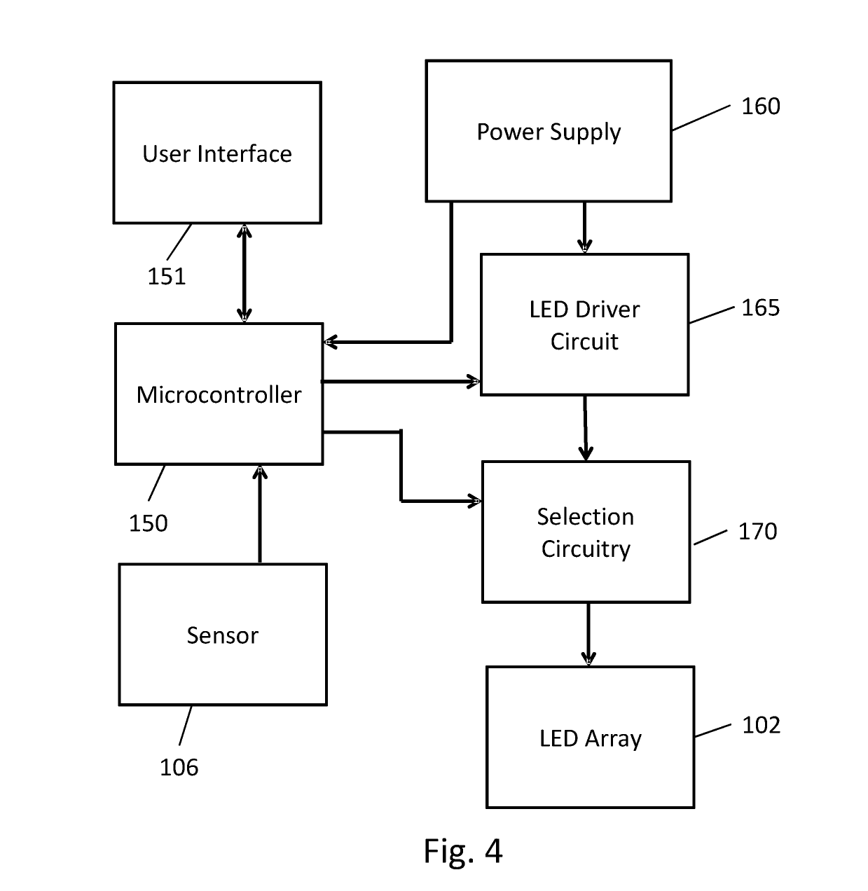

FIG. 4 shows a block diagram illustrating control circuitry used to control the operation of the device shown in FIGS. 1 and 2;

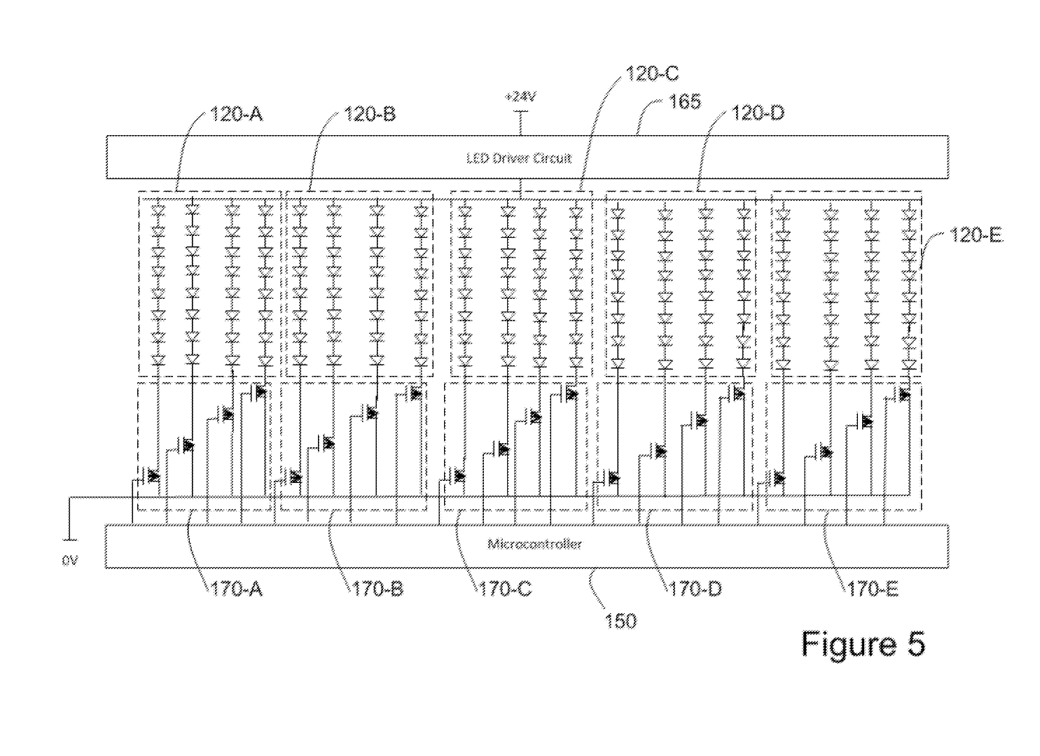

FIG. 5 is a circuit diagram illustrating driver circuitry and selection circuitry used to drive current through the LEDs and also illustrating the way in which the LEDs shown in FIG. 3 are electrically connected together in groups;

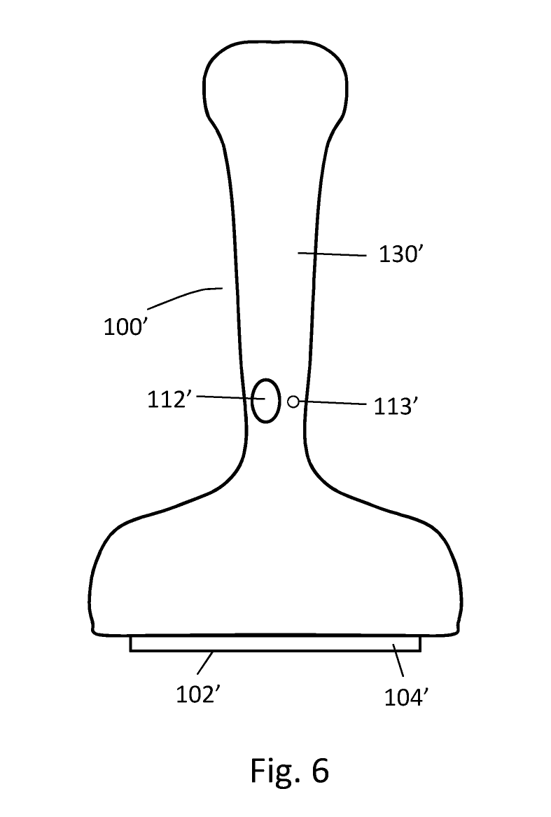

FIG. 6 is a side view of a handheld device according to a further embodiment;

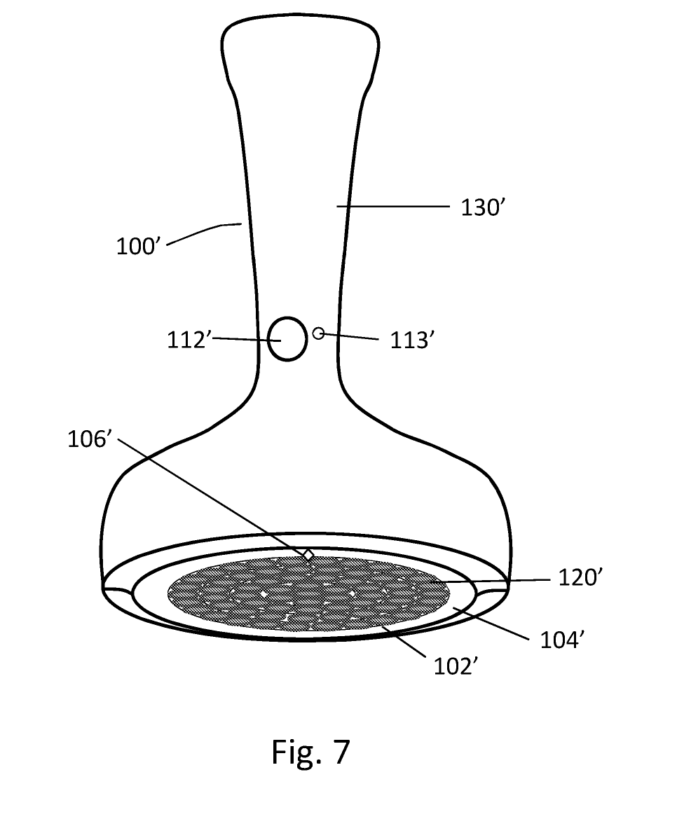

FIG. 7 is a perspective view of the device illustrated in FIG. 6;

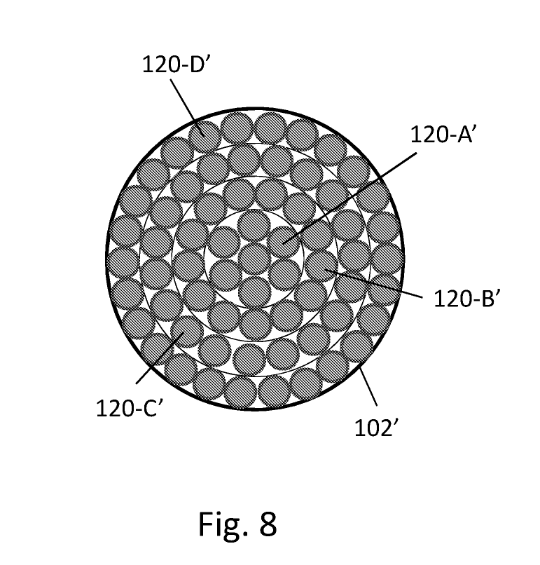

FIG. 8 illustrates an array of electromagnetic sources that forms part of the device shown in FIGS. 6 and 7;

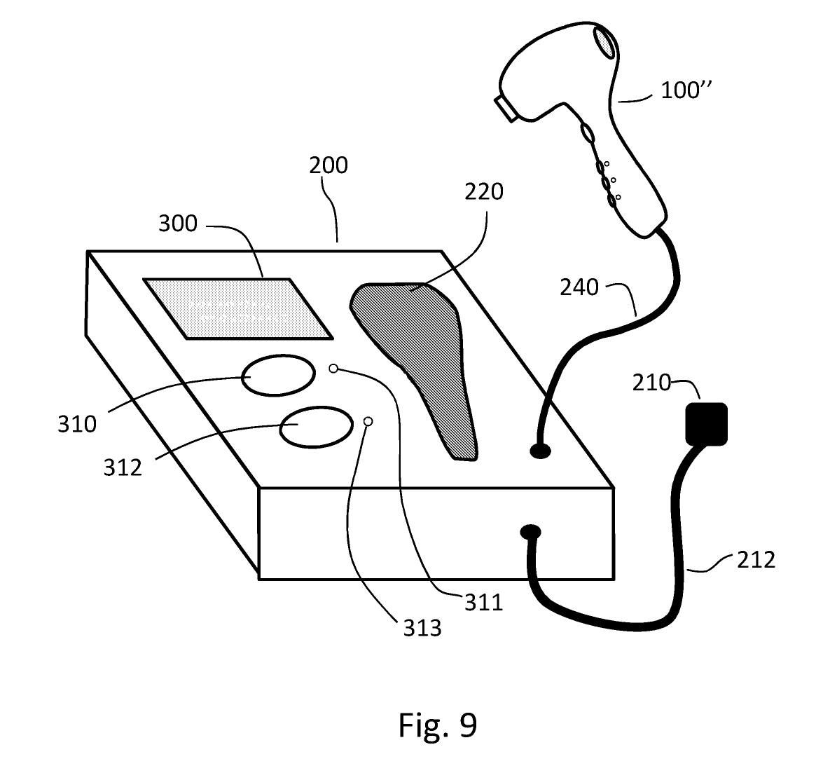

FIG. 9 is a schematic illustration of an alternative device that operates with a docking station; and

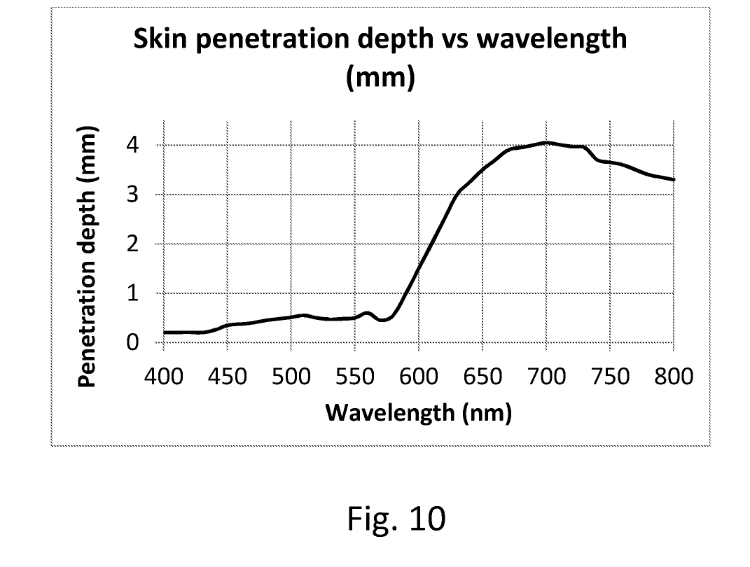

FIG. 10 is a plot illustrating the penetration depth of light energy into skin for different wavelengths.

### EMBODIMENTS

Overview and Discussion

A first embodiment that will be described below provides a device that can perform selective photothermolysis using LEDs to deliver light energy to a user's skin and hair follicles to cause damage to the hair follicle. The device is designed for home use and offers a number of advantages over existing IPL based devices. Before discussing the device of this embodiment in detail, an explanation will be given as to how an LED based device can surprisingly provide the energy density level required to perform selective photothermolysis.

Solid state light emitting diode (LED) technology has been used for a range of different lighting and display applications for many years; in fact the first visible LED was developed almost 50 years ago. Recently however, the efficiency, reliability and power density of these devices has been improved significantly. This has partly been driven by the need to reduce carbon emissions which has driven the market towards producing ever more efficient light sources. Today, white LEDs are commercially available with efficiencies in excess of 120 lumens/watt which is significantly more energy efficient than conventional incandescent light sources and comparable, if not slightly better than high efficiency fluorescent sources.

In addition to high efficiency white LED light sources, the underlying semiconductor technology, device packaging and manufacturing methods for these light sources have also seen significant technical improvements over recent years. As a result, high efficiency single colour light sources are available (blue, red, green etc.). LEDs in the red and red/orange part of the visible spectrum are available today with electrical to light conversion efficiencies of about 50% (when driven at powers of about 1 W with adequate thermal management—see e.g. the Philips Lumileds device datasheets for example DS105 and DS68).

Comparing the performance of the best performing high brightness LEDs which are currently on the market with the energy density generated from a typical home use IPL flash lamp, it would appear that the LED is unable to generate anywhere near enough energy to be useful for hair removal using selective photothermolysis. For example, a high power Lumileds Rebel LED, which generates white light in the visible spectrum from about 400 nm up to 700 nm, can be driven at about 5 W. With an electrical to optical conversion efficiency of about 30%, this would generate around 1.5 W. Assuming that this can be delivered in a pulse of about 30 ms (equivalent to a typical home use IPL device), and taking into account the package size of this device which would allow about 1 device per 15 mm², the total energy density delivered would be about 0.3 J/cm². This is more than an order of magnitude lower than is delivered by a typical home use IPL flash lamp. From this initial analysis it would appear that LED technology is not a suitable choice for hair removal using selective photothermolysis.

However, one factor which is often overlooked when comparing different light sources for hair removal, is the skin penetration depth. The skin penetration depth (as used herein) is a measure of how deep the light can penetrate the skin and is defined as the depth at which the intensity of the light inside the material falls to 1/e (about 37%) of its original value at the surface. FIG. 10 shows the skin penetration depth of light across the visible spectrum. As can be seen, light which is shorter in wavelength than about 600 nm is unable to penetrate the skin to a depth of more than about 1 mm. With the home use IPL devices currently on the market, much of the light emitted is below 600 nm and so is unable to penetrate the skin to depths of more than about 1 mm.

Taking the skin penetration depth into account, it becomes clear that a white (broadband) light source is not a good choice for selective thermolysis. If we consider an LED light source (which is a narrowband source), at a wavelength at around 650 nm or above, then this is likely to be a factor of 2 or 3 times more effective (for the same incident energy density) at inducing selective thermolysis in a hair follicle which is 1-2 mm deep in the skin, compared to the home use IPL light sources.

In addition to the benefits of better skin penetration which can be achieved by using an LED which has a wavelength at around 650 nm, LEDs in this wavelength range also have relatively high electrical to optical conversion parameters. Data from Philips Lumileds indicates that efficiencies of about 50% electrical to optical power can be achieved using today's technology. It is also likely that this conversion efficiency will see continual improvement as LED technology continues to advance over the next few years.

The latest high power LEDs available from the leading manufacturers—Philips Lumileds, Cree, Osram etc. are also capable of being driven at high currents in a pulsed mode. Whilst the LED efficiency is reduced when this is done, it is possible to drive the LEDs up to about 15 W and still achieve about 25% electrical to optical conversion efficiency.

The energy density which can be achieved with an LED array is limited by the chip packing density. Using the standard packaging supplied by the manufacturers (e.g. the Cree XP-E or Lumileds Rebel) of the best high power LEDs available today, it is difficult to achieve a packing density of better than about 1 device per 10 mm². However, the basic semiconductor chips (without their packaging) are also available from the manufacturers and by building a custom LED array, using a chip-on-board design (in which the semiconductor chips are mounted directly onto a circuit board or onto a heatsink mounted on the circuit board), it is possible to improve the packing density of the LED chips to around 1 device per 6 mm². Higher packing densities are also expected in the future as improvements are made to the underlying semiconductor manufacturing process.

In doing this, it becomes possible to develop a custom LED array of red or red/orange emitters which have the following characteristics:

| LED Pulse power | 15 | W |
|---|---|---|
| Pulse width | 30 | ms |
| Efficiency | 25% | |
| LED footprint | 6 | mm² |
| Energy density | 1.8 | J/cm² |

The energy density of this LED array is still less than that which is used in the home use IPL products. However, considering the impact of skin penetration depth, and the choice of a single colour red or red/orange LED rather than a broadband white light source, the 1.8 J/cm² is broadly equivalent to the useful energy obtained from existing home use IPL devices which emit broadband light with an energy density of about 5-8 J/cm². As a result, the LED based device can deliver the same efficacy of light dose for selective photothermolysis as the home use IPL devices currently on the market.

It is also possible to consider a custom LED array which has slightly different parameters from those given in the table above. For example, an LED array may be provided which is driven at lower power levels—typically 10 W—which enables slightly higher efficiency to be achieved (˜35%). In addition, if LEDs such as the Luxeon Z device (available from Philips Lumileds) are used, then it is possible to achieve a packing density of around 1 device per 3 mm². In doing this, it becomes possible to develop a custom LED array of red or red/orange emitters which have the following characteristics:

| LED Pulse power | 10 | W |
|---|---|---|
| Pulse width | 30 | ms |
| Efficiency | 35% | |
| LED footprint | 3 | mm² |
| Energy density | 3.5 | J/cm² |

As with the previous example, the energy density here, although higher, is still slightly less than that which is used in the home use IPL products, but after factoring in the impact of skin penetration depth and the choice of a narrow band light source (rather than broadband) it can be seen that the LED based device can deliver the same efficacy of light dose for selective photothermolysis as the home use IPL devices.

One further benefit of using LEDs for this application should also be noted. Unlike IPL flash tubes which produce light in a single linear source, an LED array can be configured so that individual groups of LEDs can be switched on and off independently. In addition, due to its efficient generation of light, and better thermal management (the LED array can be bonded directly to a heatsink), LEDs need no recovery time. This is different from the IPL flash lamps which will not sustain continuous flashing due to the fact that the gas and the material within the flash tube needs to be allowed to cool down for a certain period of time before another flash can be generated. As a result, the delay between consecutive flashes from an LED array is only limited by the relaxation time of the hair follicle. As discussed earlier, this is typically in the range of 30-50 ms. If the LED array is flashed every 200 ms, 5-15 times more flashes per second can be delivered from the LED device than from an IPL flash lamp. This has the potential to significantly increase the efficacy of the LED based device for hair removal using selective photothermolysis.

It should also be noted that the LED device has the following additional benefits compared to IPL devices:
Potential for lower cost—LED drive electronics are relatively straight forward and simple in comparison to the relatively expensive drivers required for IPL flash lamps
The LED array can be positioned very close to the user's skin—as there is no need for additional optical components—again this is likely to reduce the cost of the LED device in comparison to the available IPL devices which all need optics to generate a uniform illumination profile from a linear tube
The LED lifetime is typically 50,000 hours—significantly more than a flash tube (which is probably around 50 hours maximum)—which means the LED product will last significantly longer

A discussion will now be given of the LED based device used in this first embodiment. FIG. 1 is a side view of a handheld device 100 for providing energy to a surface of the user's skin. As shown, there is a handle 130, for the user to hold the device 100. There is an array 102 of light sources (which in this embodiment are light emitting diodes) that is surrounded by a lip 104 to provide contact to the skin surface during use. There is also provided one or more sensors 106 on the edge of the lip 104 to monitor, for example, the temperature of the array or shade of the skin surface. Buttons 110, 112, 114, 116 are also provided for switching the device on and off and for controlling certain functions of the device, such as the energy intensity, wavelength of the radiation or length of the pulses. There are further provided indicator lights 113, 115, 117 to indicate the functions of the device that have been selected. An air inlet 140 is also provided to allow for the ambient air to cool the heatsink (not shown) on which the array 102 of LEDs is mounted. A fan may be provided in some embodiments to draw the air into the air inlet 140 to provide forced air cooling of the heatsink and LEDs, although in many embodiments such forced air cooling is not essential.

Reference is now made to FIG. 2, which shows a front view of the device 100 of FIG. 1. During use, a small gap is provided between the surface of the LEDs 120 and the user's skin. This gap is provided by the lip 104, which protrudes slightly beyond the front surface of the array 102 and protects the user's skin from the surface of the LEDs which can get hot during use. If desired, a transparent window, for example made of glass, may be provided in this gap to protect the LEDs. However, the lip 104 is not essential and in some embodiments the user's skin may be allowed to contact the surface of the LEDs directly. One of the advantages of using LEDs is that they can be positioned very close to the surface of the skin as there is no need for additional optical components between the LEDs and the user's skin.

Reference is now made to FIG. 3, which shows a closer view of the array 102 of LEDs used in this embodiment. As shown, the array 102 is rectangular in shape and comprises a grid of 8×20 LEDs 120. The LEDs are packed to a density of around 1 LED per 6 mm². This is achieved by using a chip-on-board design—in which the LEDs (without their own packaging) are packed next to each other on a circuit board substrate (or in this embodiment on a heatsink (not shown) mounted on the circuit board). The area covered by the array 102 is therefore approximately 10 cm², which allows the user to treat an area of skin that is also approximately 10 cm². In a preferred embodiment, the LEDs emit radiation having a wavelength that is longer than 600 nm and shorter than 900 nm, and most preferably having a wavelength around 650 nm (due to the wide availability of LEDs operating at this wavelength and their efficient electrical to optical conversion efficiency). This allows much deeper penetration, beyond 1 mm, of radiation into the surface of the skin compared to radiation at shorter wavelengths. As there are no focussing optics between the LEDs 120 and the user's skin, the light energy from the array of LEDs provides a substantially uniform energy density over the surface of the user's skin immediately adjacent the LEDs that are powered. In this case substantially uniform means a variation of no more than +−30%, and preferably no more than +−10%. Typically, in this embodiment, this energy density will be about 1.8 J/cm², which is sufficient for selective photothermolysis.

In this embodiment, the LEDs are driven to emit pulses of light radiation having a pulse duration of approximately 30 ms; and the time between pulses is approximately 200 ms (which is greater than the thermal relaxation time constant of a hair follicle). Although the array 102 has 160 LEDs, in order to reduce the likelihood of the device overheating, the LEDs are not all driven at the same time. More specifically, in this embodiment and as illustrated in FIG. 3, the array of LEDs 120 is divided into five groups (Groups A to E, labelled 120-A to 120-E) and the LEDs in the different groups are driven at different times. Preferably, the LEDs in physically adjacent groups are not driven consecutively—again to help with heat dissipation. Thus, for example, the LEDs in group A may be pulsed first for 30 ms; followed by the LEDs in group C for 30 ms; followed by the LEDs in group E for 30 ms; followed by the LEDs in group B for 30 ms; followed by the LEDs in group D for 30 ms. The sequence then restarts by pulsing the LEDs in group A. By continuously cycling through this driving sequence and by pulsing the LEDs in each group for approximately 30 ms with a short gap (which can be as small as 1 μs, but which in this embodiment is about 10 ms) between the driving of the LEDs in the different groups (to allow switching the selection circuitry described below), the LEDs in each group can be pulsed approximately once every 200 ms. Since adjacent groups of LEDs are not driven consecutively, the heat generated by driving the LEDs in one group is better able to be dissipated before the same LEDs are driven again.

Reference is now made to FIG. 4, which illustrates schematically circuitry that is used to control the operation of the device 100. As shown, a microcontroller 150 is provided for controlling the operation of the device 100 and in particular for controlling the driving of the LEDs 120 in response to user inputs entered via a user interface 151. In this embodiment, the user interface 151 comprises the above described buttons 110, 112, 114 and 116 and indicator lights 113, 115 and 117. The microcontroller 150 is responsive to the user inputs to control the operation of the device 100, for example, to vary the duration that each group of LEDs is pulsed or to vary the wavelength of the radiation emitted by the LEDs. A power supply circuit 160 provides electrical energy for powering the microcontroller 150 and for providing the electrical energy required to drive the LEDs in the array 102. Typically the power supply circuit 160 will comprise one or more batteries, although in some embodiments, power may be provided to the hand held device 100 from a mains supply, in which case, the power supply circuit will perform the required AC to DC conversions to provide the DC power rails for the microcontroller 150 and the LEDs 120. An LED driver circuit 165 is provided for driving the LEDs; and selection circuitry 170 is provided that is controlled by the microcontroller 150 and that can select a group of LEDs to be driven at any point in time. As discussed above, one or more sensors 106 are provided that can monitor the temperature of the array 102 and/or that can sense the shade of the user's skin surface. The signals from the sensor(s) 106 are provided to the microcontroller 150, which may stop the driving of the LED array 102 if the sensor 106 indicates that the array 102 is overheating; or that may vary the way in which the LEDs 120 are driven depending on the shade of the user's skin to achieve optimal performance of the device for the particular user.

FIG. 5 illustrates in more detail the form of the LED driver circuit 165, the LED array 102 and the selection circuitry 170. The LED driver circuit 165 may comprise a number of standard driver circuits (such as the LT3791 provided by Linear Technology) that can generate the desired driving signals for the LEDs 120. The LEDs are connected in 20 strings of eight LEDs and the driver circuit 165 generates the driving signals from a 24 Volt rail provided by the power supply circuit 160. FIG. 5 also shows the selection circuitry 170 used to control which group of LEDs is driven at any given point in time. In particular, the selection circuitry includes five groups of MOSFET switches 170-A to 170-E, that are controlled by the microcontroller 150. When the LEDs in group A 120-A are to be driven, the microcontroller 150 closes switches 170-A and opens the other switches, thereby allowing current to flow only through the LEDs in group A to ground. Similarly, when the LEDs in group B 120-B are to be driven, the microcontroller 150 closes switches 170-B and opens the other switches; when the LEDs in group C 120-C are to be driven, the microcontroller 150 closes switches 170-C and opens the other switches; when the LEDs in group D 120-D are to be driven, the microcontroller 150 closes switches 170-D and opens the other switches; and when the LEDs in group E 120-E are to be driven, the microcontroller 150 closes switches 170-E and opens the other switches. In this way, driving current is only able to flow through the LEDs in the selected group and so only the LEDs in the selected group will generate light.

While a specific embodiment has been described above, others may be provided. In particular, the housing shape may vary significantly without altering the functioning of the device 100. For example, FIG. 7 shows an alternative handheld device 100′ for providing energy to a surface of the user's skin according to an alternative embodiment. In this embodiment, the axis of the handle 130′, for the user to hold the device 100, is substantially perpendicular to the plane of the array 102′ of electromagnetic (EM) energy sources. The array 102′ is again surrounded by a lip 104′ to provide contact to the skin surface. A button 112′ is provided for switching the device on and off and for controlling certain functions of the device, such as the energy intensity, wavelength of the radiation or length of the pulses etc. An indicator light 113′ is also provided to indicate the functions of the device that have been selected.

FIG. 8, shows an alternative view of the device 100′ shown in FIG. 7, showing more detail of the array 102′ of light sources 120′. FIG. 8 also shows that a sensor 106′ is provided on the edge of the lip 104′ to monitor, for example, the temperature or shade of the skin surface. As shown in FIG. 8, in this embodiment, the array 102 is a circular array of light sources 120′ which may be driven in different groups as per the first embodiment described above. FIG. 9 illustrates the way in which the circular array of light sources can be divided into 4 different circular groups 120-A to 120-D. Alternatively, the sources could be divided into segments to maximise the energy density generated by each segment.

In the above embodiments, the device 100 was a self-contained hand held device that was battery or mains operated. FIG. 10 illustrates an alternative embodiment, in which the hand held device 100″ is configured to operate with a docking station 200. As shown, the station 200 includes a dock 220, in which the hand held device 100″ can be placed while not in use. If battery powered, the internal battery of the hand held device may be charged when the device is docked. In this example, however, the hand held device 100″ is powered by an AC power cord 240 connected to the docking station 200. The docking station 200 itself is configured to connect to an AC mains power supply by means of a plug 210 and power cable 212. The docking station 200 includes a user interface display 300 that can display information about the configuration and settings of the hand held device 100. The docking station 200 also has a number of buttons 310, 312 for the user to select certain settings, controls and preferences for use of the hand held device 100″. Indicator lights 311, 312 are also be provided in order to indicate which settings or preferences have been selected.

It will be understood that the present invention has been described above purely by way of example, and modification of detail can be made within the scope of the invention. For example, whilst the LEDs were grouped into different groups and each group was selected for driving at different times, multiple groups may be selected for driving at the same time or in overlapping time periods, if desired.

In the embodiment described above, an array of light sources in the form of LEDs was used. In alternative embodiments, other types of light sources may be used, such as laser diodes, VCSELs (vertical-cavity surface emitting lasers), superluminescent diodes etc. Like the LEDs, these light sources are semiconductor devices that can be arranged in an array (with a packing density that is typically between one source every 15 mm² and one source every 2 mm²) and can provide light energy to the surface of the user's skin at an energy density greater than 0.5 J/cm² over a relatively wide area (typically at least 1 cm² and preferably about 10 cm²) and without the use of focussing optics or the like. They are also, therefore, suitable for use in hand held devices for home use in treating areas of user's skin.

Arrays of such semiconductor light sources preferably emit energy at an energy density greater than 1.5 J/cm² and preferably greater than 1.8 J/cm². An upper limit on the energy density of about 5 J/cm² can be provided to avoid burning of the user's skin.

The area of the user's skin that can be treated is limited only by the overall size of the array of light sources. If the array is too small, then the device will not be attractive to users as it will take a long time to treat a desired area of their skin. However, if the array is too large, then the device will become too costly, again making the device less attractive to users. The device described above used an array that was approximately 10 cm². This represents a practical compromise between the cost of the device and the area that can be treated by the device. Other sizes are of course possible—including arrays that can treat an area of about 1 cm² to arrays that can treat an area of about 50 cm².

Each feature disclosed in the description, and (where appropriate) the claims and drawings may be provided independently or in any appropriate combination.

1. A device for providing energy to the skin of a user, the device comprising:
an array of semiconductor light sources;
a power supply circuit for providing electrical energy to the semiconductor light sources; and
a controller for controlling the operation of the semiconductor light sources, to cause the semiconductor light sources to emit light in pulses; and wherein:
the array of semiconductor light sources and the power supply circuit are configured such that the array of semiconductor light sources emit light at an energy density greater than 0.5 J/cm².

2. The device of claim 1, wherein:
the array of semiconductor light sources and the power supply circuit are arranged such that the energy density of the light emitted by the array is substantially uniform over the user's skin.

3. The device of claim 1, wherein:
the array of semiconductor light sources is arranged to emit light over an area between 1 cm² and 100 cm².

4. The device of claim 1, wherein:
each semiconductor light source is configured to emit single wavelength light or narrow-band light having a bandwidth between 1 nm and 100 nm.

5. The device of claim 1, wherein:
the semiconductor light sources comprise light emitting diodes (LEDs).

6. The device of claim 1, wherein:
the semiconductor light sources are operable to emit light having a skin penetration depth of more than 1 mm, and preferably more than 2 mm.

7. The device of claim 1, wherein:
the semiconductor light sources are operable to emit light having a wavelength that is longer than 600 nm, and preferably longer than 650 nm.

8. The device of claim 1, wherein:
the semiconductor light sources are operable to emit light having a wavelength that is shorter than 900 nm, and preferably shorter than 750 nm.

9. The device of claim 1, wherein:
the semiconductor light sources are operable to emit light having a wavelength of approximately 650 nm.

10. The device of claim 1, wherein:
the array of semiconductor light sources and the power supply circuit are configured such that the array of semiconductor light sources emit light at an energy density greater than 1.5 J/cm².

11. The device of claim 1, wherein:
the array of semiconductor light sources and the power supply circuit are configured such that the array of semiconductor light sources emits light at an energy density of approximately 1.8 J/cm².

12. The device of claim 1, wherein:
the array of semiconductor light sources and the power supply circuit are configured such that the array of semiconductor light sources emits light at an energy density less than 5 J/cm².

13. The device of claim 1, wherein:
the packing density of the semiconductor light sources is greater than 1 source per 15 mm² and less than 1 source per 2 mm².

14. The device of claim 1, wherein:
the array of semiconductor light sources is arranged in a plurality of groups and wherein the controller is arranged to control the driving of the semiconductor light sources in each group so that the semiconductor light sources in the different groups are driven during different time intervals.

15. The device of claim 14, wherein:
the controller is arranged to control the driving of the semiconductor light sources so that no two adjacent groups emit light sequentially.

16. The device of claim 1, wherein:
the controller is arranged to control the driving of the semiconductor light sources so that semiconductor light sources are driven periodically, preferably approximately every 200 ms.

17. The device of claim 1, wherein:
the controller is arranged to control the driving of the semiconductor light sources so that the semiconductor light sources are driven to emit pulses of radiation having a pulse duration of approximately 30 to 50 ms.

18. The device of claim 1, further comprising:
a sensor arranged to monitor at least one of the temperature of the skin surface and the shade of the skin surface.

19. The device of claim 1, wherein the device is handheld.

20. The device of claim 1, wherein no focussing optical element is provided in front of the array of light sources.

21. The device of claim 1, wherein:
the energy provided to the skin of a user is suitable for hair removal by selective photothermolysis.

22. A device for providing energy to the skin of a user, the device comprising:
an array of light sources arranged in a plurality of groups;
selection circuitry for selecting a group of light sources;
a power supply circuit for providing electrical energy to the selected group of light sources; and
a controller for controlling the selection circuitry to select different groups of the light sources at different times so that different groups of energy sources receive power from the power supply circuit during different time intervals.

23-26. (canceled)

27. The device of claim 22, wherein:
each light source comprises a light emitting diode (LED).

28-31. (canceled)

32. The device of claim 22, wherein:
each group of light sources is configured to emit light at an energy density greater than 1.5 J/cm² and less than 5 J/cm².

33-34. (canceled)

35. The device of claim 22, wherein:
the packing density of the light sources is less than 1 source per 15 mm² and greater than 1 source per 2 mm².

36. The device of claim 22, wherein:
the controller is arranged to control the driving of the light sources so that no two adjacent groups emit radiation sequentially.

37. The device of claim 22, wherein:
the controller is arranged to control the driving of the light sources so that light sources are driven periodically, preferably approximately every 200 ms.

38. The device of claim 22, wherein:
the controller is arranged to control the driving of the light sources so that the light sources are driven to emit pulses of radiation having a pulse duration of approximately 30 to 50 ms.

39-40. (canceled)

41. The device according to claim 22, wherein:
each group of light sources comprises at least one string of serially connected light sources.

42. The device of claim 22, wherein no focussing optical element is provided in front of the array of light sources.

43. (canceled)

44. A method of providing energy to a surface of the skin, the method comprising:
providing an array of semiconductor light sources;
providing electrical energy to the semiconductor light sources;
controlling the operation of the semiconductor light sources, causing them to emit light in pulses at an energy density greater than 0.5 J/cm²; and
directing the radiation emitted from the EM energy sources towards the surface of the skin.

45. (canceled)
